(19) United States
(12) Patent Application Publication
(10) Pub. No.: US 2003/0104623 A1
(43) Pub. Date: Jun. 5, 2003

(54) Method of introducing a protein into cells

(75) Inventors: Hidenori Yamada, Okayama (JP); Junichiro Futami, Hyogo (JP); Hidetaka Nakanishi, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Nippon Shokubai Co., Ltd., Osaka (JP)

(21) Appl. No.: 10/156,888

(22) Filed: May 30, 2002

(30) Foreign Application Priority Data
Nov. 29, 2001 (JP) 2001-363967

(51) Int. Cl. C12N 5/02; C12N 15/85; A61K 9/14

(52) U.S. Cl. 435/455; 435/325; 424/486

(57) ABSTRACT

A conjugate which enables a protein or peptide to be introduced into cells and a method for introducing the protein or peptide into cells using the conjugate with time and amount controllability and efficiency.

### TECHNICAL FIELD

The present invention relates to a novel protein conjugate and a method for efficiently introducing a protein into cells using the conjugate.

### BACKGROUND ART

Time signals of expression or modification of a protein engaged in proliferation, differentiation or development of various cells are now being clarified. When applying these findings to engineering fields such as regeneration medicine or tissue engineering, if it is possible to control existence of a protein intended to function in cells at a designated amount for a designated period, it is expected to broaden various potentialities.

As a technique to enable a designated protein to function in cells, gene transfer is almost the only method at present. When constant functioning of the designated protein in cells is desired, gene transfer is effective. However, when the designated protein is desired to transiently function in cells, it is preferable to introduce the protein itself into the cells.

Conventionally, as a method for introducing a protein itself into cells, because of the necessity of traverse through cell membranes, examples of methods to be used include a special method such as microinjection, and a method wherein a protein is encapsulated into a capsule shaped-material such as a liposome and the capsule shaped-material is fused with the cell membrane so as to introduce the content (protein etc.) of the capsule shaped-material into the cell. In addition, though kinds of cells are limited, another method for introducing a protein into cells through a receptor-dependent route has come into practical use, wherein various receptors expressed on cell surfaces are targeted and ligands of these receptors are used as carriers.

The inventors have recently confirmed that apart from these routes, a highly cationic protein or a protein cationized by chemical modification was electrostatically adsorbed to a negatively charged cell surface, and incorporated into cells with high efficiency (Futami et al., Biochemistry, 40, 7518-7524, 2001). Likewise, recently it has been reported that a protein to which a highly basic TAT peptide derived from HIV (Schwarze et al., Science, 285, 1569-1572, 1999) or a cationic peptide (Futaki et al., J. Biol. Chem., 276, 5836-5840, 2001) such as Poly-Arg is added, can efficiently cross a cell membrane. Although in all the cases mentioned above, mechanisms have yet to be known in detail, a cell membrane crossing route mediated by the electrostatic interaction between a cationic protein and a cell surface is supposed.

However, a conventional protein cationization method requires modification of many amino acid side chains in a protein molecule, thereby deteriorating its function.

### DISCLOSURE OF THE INVENTION

An object of the present invention is to provide a conjugate which enables a protein to be introduced into cells, and a method for introducing the protein into cells using the conjugate with time and amount controllability and efficiency.

The present inventors have conducted studies to solve the above-described problems. As a result, the inventors have found that the problems can be solved by combining a polyalkyleneimine such as polyethyleneimine which is a cationic polymer, with a protein to cationize the protein, thereby accomplishing the present invention.

Namely, the present invention includes the following:

(1)A method for transducing a protein or peptide into a cell, comprising a step of transporting the protein or peptide into the cell by using a conjugate formed by binding the protein or peptide with a polymer having a cation value of more than 2 and no more than 30,000.

(2) The method according to (1) above wherein the polymer is a polymer having a polyalkylenepolyamine skeleton, a polyallylamine skeleton, a polyvinylamine skeleton, a poly(dialkylaminoalkyl (meth)acrylate) skeleton, a poly(meth) acrylic dialkylaminoalkylamide skeleton, a polyamidine skeleton, a polyvinylpyridine skeleton or polyvinylimidazole skeleton, or a salt thereof.

(3) The method according to (1) or (2) above wherein the polymer has a number average molecular weight within a range of from 100 to 1,000,000.

(4) The method according to any of (1) to (3) above wherein the conjugate is formed by binding the protein or peptide to the polymer via covalent bond.

(5) The method according to any of (1) to (4) above wherein the conjugate is formed by binding the protein or peptide to the polymer via an amide bond, disulfide bond or thioether bond.

(6) A method for transducing a protein or peptide into a cell, comprising a step of transporting the protein or peptide into the cell by using a conjugate formed by binding the protein or peptide with a polymer having a polyalkylenepolyamine skeleton.

(7) The method according to (6) above wherein the polymer is polyalkyleneimine.

(8) The method according to (6) or (7) above wherein the polymer is polyethyleneimine.

(9) The method according to any of (6) to (8) above wherein the polymer has a number average molecular weight within a range of from 100 to 1,000,000.

(10) The method according to any of (6) to (9) above wherein the polymer has a number average molecular weight within a range of from 100 to 100,000.

(11) The method according to any of (6) to (10) above wherein the conjugate is formed by binding the protein or peptide with the polymer via covalent bond.

(12) The method according to any of (6) to (11) above wherein the conjugate is formed by binding the protein or peptide with the polymer via an amide bond, disulfide bond or thioether bond.

(13) A conjugate formed by binding a protein or a peptide with a polymer having a cation value of more than 2 and no more than 30,000, and having a number average molecular weight within a range of from 100 to 1,000,000.

(14) The conjugate according to (13) above wherein the polymer is a polymer having a polyalkylenepolyamine skeleton, a polyallylamine skeleton, a polyvinylamine skeleton, a poly(dialkylaminoalkyl (meth)acrylate) skeleton, a poly(meth) acrylic dialkylaminoalkylamide skeleton, a polyamidine skeleton, a polyvinylpyridine skeleton or polyvinylimidazole skeleton, or a salt thereof.

Further, the present invention includes the following:

(1) A conjugate formed by binding a protein with a polyalkyleneimine;

(2) The conjugate according to (1) above wherein the polyalkyleneimine is polyethyleneimine;

(3) The conjugate according to (1) or (2) above wherein the polyalkyleneimine has a number average molecular weight within a range of from 100 to 100,000;

(4) The conjugate according to any one of (1) to (3) above wherein the protein is bound with the polyalkyleneimine via any one of an amide bond, disulfide bond, and thioether bond; and

(5) A method for transducing a protein into a cell, comprising a step of transporting the protein into the cell by using the conjugate according to any one of the above (1) to (4).

### DETAILED DESCRIPTION OF THE INVENTION

The present invention will hereinafter be described in detail.

In this specification, “a protein or peptide” is defined as a compound produced by binding 2 or more amino acids with each other via a peptide bond. A protein or peptide to be used in the present invention is not limited, and any protein or peptide such as a peptide, an enzyme, or a protein having other functionality (physiological activity) can be used and the molecular weight thereof is preferably within a range of from 100 to 1,000,000. Incidentally, the term “protein” in this specification includes a conjugated protein formed by conjugation of a sugar chain, lipid and/or a phosphate group. Further, a structure of the protein may either be a native state or a denatured state.

A polymer to be used in the present invention which has a cationic group may be e.g. a polymer having a cation value of more than 2 and no more than 30,000. “Cation value” in this specification is defined as a value obtained by dividing the product of the amine value (mmol/g) and the number average molecular weight of the polymer by 1000. The cation value of the polymer to be used in the present invention is in general more than 2 and no more than 30,000, preferably more than 2 and no more than 20,000, more preferably more than 2 and no more than 2,500, in particular preferably more than 2 and no more than 250, and most preferably from 4 to 70. Incidentally, “amine value (mmol/g)” is an indicator of the total amine in a sample compound and it is represented as mmol number of amino groups that exist in 1 g of the sample compound. The amine value of the sample compound may be measured according to a known method for quantitative analysis of amino group. Such quantitative analysis of amino group includes a method described in “Shin-jikkenn Kagaku Koza Vol. 13 Yuki-Kagaku-Kozo I” pp. 88-99 (MARUZEN Co., Ltd., edit. by the Chemical Society of Japan; pub. in Nov. 20, 1978) and colloidal titration method. Colloidal titration is described in “Colloidal titration method 1st Ed.” (Nankodo Co., Ltd., edit. by R. Senju; pub. in Nov. 20, 1969). A suitable quantitative method should be chosen for accurate measurement of amine value, in consideration of a form and solubility of the sample compound, and contaminants in the sample. The amine value of the polymer to be used in the present invention is not particularly limited, but it is preferably within a range of from 1 to 30, more preferably 5 to 25.

Moreover, the polymer to be used in the present invention has a number average molecular weight, in general, within a range of from 100 to 1,000,000, preferably 100 to 100,000, more preferably 100 to 10,000, most preferably 200 to 3,000. In passing, for determining the number average molecular weight of the polymer, when the number average molecular weight of the polymer is 10,000 or less, ebullioscopic method is employed. When the number average molecular weight thereof exceeds 10,000, GPC is employed for accurate determination of molecular weight.

Examples of the polymer to be used in the present invention include a polymer having a polyalkylenepolyamine skeleton, a polyallylamine skeleton, a polyvinylamine skeleton, a poly(dialkylaminoalkyl (meth)acrylate) skeleton, a poly(meth) acrylic dialkylaminoalkylamide skeleton, a polyamidine skeleton, a polyvinylpyridine skeleton or polyvinylimidazole skeleton, and a co-polymer thereof. Further, a salt of these polymers e.g. primary, secondary, tertiary, or quarternary ammonium salt may be used as well. Additionally, those polymers, which are chemically altered or modified, may be used.

Specific examples of these polymers include polyalkylenepolyamine (e.g. polyalkyleneimine (polyethyleneimine, polypropyleneimine)), polyallylamine (e.g. polyallylamine, polydiallyldimethylammonium chloride), Hofmann decomposition product of polyacrylamide, polyvinylamine (e.g. a hydrolysate of polyvinylacetamide, a hydrolysate of polyvinylphthalimide, a hydrolysate of N-vinylformamide), dialkylaminoalkyl(meth)acrylamide (co)polymer (e.g. dimethyl-aminopropyl(meth)acrylamide (co)polymer), dialkylaminoalkyl(meth)acrylate (co)polymer (e.g. polymethacryloyloxyethyl trimethylammonium chloride), polyamidine, polyvinylpyridine, polyvinylimidazole, a dicyandiamide condensate, an epichlorohydrin-dialkylamine condensate (e.g. an epichlorohydrin-dimethylamine condensate), a dialkylamine-alkyldihalide condensate (e.g. a dimethylamine-ethylenedichloride condensate, polyvinylimidazoline, polyvinylbenzyl trimethylammonium chloride, carboxy methyl cellulose quarternary ammonium (quarternary ammonium CMC), glycolchitosan, cationized starch and the like.

Following is the theoretical amine values of typical polymers among the above-mentioned polymers. The theoretical amine values below are calculated by multiplying a reciprocal of molecular weight of a component monomer by 1,000. Generally, the amine value experimentally determined by the above-mentioned method is almost identical with the theoretical value within a range of measurement errors. The cation value of the polymer may be calculated on the basis of the amine value determined by the above-mentioned method. The amine value may be varied by changing a polymerization method, or copolymerizing with other components, or chemical modification of the polymer.

| | Amine value |
|---|---|
| | (mmol/g)* |
| polyethyleneimine | 23 |
| polyvinylamine | 23 |
| polyallylamine | 17 |
| polydiallyldimethylammonium chrolide | 6.2 |
| polymethacryloyloxyethyl trimethylammonium chloride | 4.8 |
| polymethacryloylaminopropyl trimethylammonium chloride | 4.5 |
| polyamidine | 6.0 |
| polyvinylbenzyl tri-methylammonium chloride | 6.3 |
| polyvinylpyridine | 10 |
| polyvinylimidazoline | 11 |
| epichlorohydrin-dimethylamine condensate | 7.2 |
| dimethylamine-ethylenedichloride condensate | 9.3 |

*theoretically maximum value

Hereinafter, a method for preparing the conjugate of the present invention will be described. When, for example, polyalkyleneimine is used as the polymer, the conjugate of the present invention is prepared as follows.

Examples of the polyalkyleneimine to be used in the present invention may be represented by the following general formula (I), and may be either linear or branched.

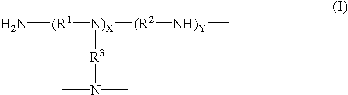

wherein each of R¹, R² and R³ represents an alkylene group, X and Y are each an integer of 0 or more, and the sum of X and Y is 1 or more.)

The polyalkyleneimine is preferably a polyalkyleneimine wherein each of R¹, R² and R³ in the above formula (I) is independently a C₂-C₄ alkylene group, and more preferably a polyethyleneimine wherein each of R¹, R² and R³ in the above formula (I) is an ethylene group.

Hereinafter, the present invention is described in a case where polyethyleneimine is used, but the present invention is not limited thereto. Besides this, other polyalkyleneimines can be used in the same manner as below.

The polyethyleneimine (hereinafter referred to as “PEI”) which is preferably used in the present invention, is represented by the following formula.

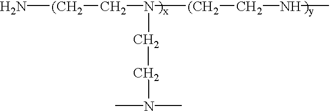

wherein X and Y are each an integer of 1 or more.

PEI is a water soluble polymer having a large positive charge density. In passing, PEI is used as a food additive such as a precipitant for fish sausage, and therefore safety in living bodies has been verified.

In the present invention, although either a PEI having a linear chain or a PEI having a branched structure with many branch chains can be used, a PEI having a branched structure represented by the following formula is preferable due to its high positive charge density. Additionally, in consideration of cell introduction efficiency, handleability etc., the PEI preferably has a number average molecular weight of 100 to 100,000, more preferably 100 to 10,000, and further more preferably 200 to 3,000. Such a low molecular weight PEI is particularly preferable.

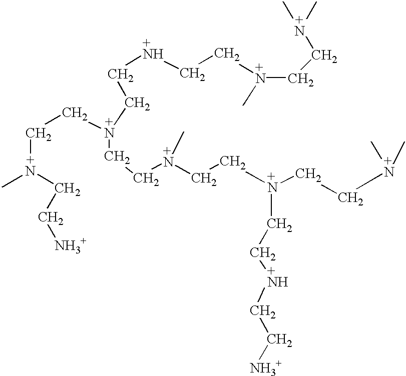

A conjugate of the present invention is formed by binding the protein to the PEI. Herein the term “binding” means a chemical bond by covalent bonding. The protein and the PEI may be directly bound without any intermediate therebetween. Alternatively, by use of a known bifunctional crosslinking reagent etc., they may be bound via an intermediate like a spacer. Although the number of PEI molecules to be bound to one molecule protein is not limited, the number is preferably 1 to 10, more preferably 1 to 3. Further, the number of binding sites for each PEI to the protein is preferably one. In the present invention, a conjugate having one PEI bound to one protein at one binding site is particularly preferable.

The binding between the protein and the PEI is not particularly limited, as long as it can bind the protein to the PEI by covalent bond. The binding is possible by various binding methods by use of known synthesis methods in the chemical field. Hereinafter the binding method of the protein to the PEI will be exemplified, but the binding method to be used in the present invention is not limited thereto.

When the protein and the PEI are bound via an amide bond, an amide bond can be formed between an amino group of the PIE and an asparatic acid residue, a glutamic acid residue, or a carboxyl group of C-terminal in the protein molecule by using an activator such as EDC (1-ethyl-3-(3-dimethylaminopropyl)-carbodiimide hydrochloride) and DCC (N,N-dicyclohexylcarbodiimide). An example case wherein EDC is used is schematically shown below.

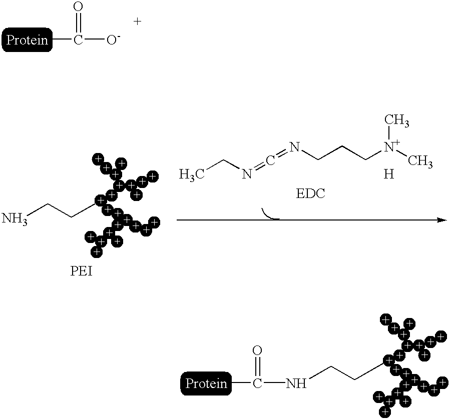

When the protein and the PEI are bound via a disulfide bond, there can be formed a covalent bond including disulfide bond between an amino group of the PEI and a thiol group of a cysteine residue in the protein molecule by using a reagent such as SPDP (N-succinimidyl-3-(2-pyridyldithio)propionate). An example case wherein SPDP is used is schematically shown below.

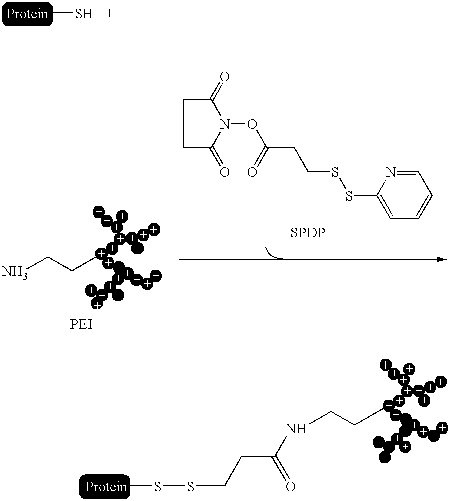

Disulfide bond is reversible, and it dissociates under reducing conditions in the cytoplasm. Therefore, when the conjugate formed by disulfide bond is introduced into cells, the PEI is separated from the protein and thus the protein is expected to exhibit its function advantageously in cells.

Other examples for binding between the protein and PEI include a method for binding an amino group of the PEI to a lysine residue or an amino group of N-terminal in the protein molecule using 2-iminothiolane etc., and a method for forming a covalent bond including thioether bond between an amino group of the PEI and a thiol group of a cysteine residue in the protein molecule using GMBS (N-(4-maleimidebutyryloxy)succinimide) etc.

In addition to the binding method mentioned above, ether bond, ester bond, imide bond, carbon-carbon bond, amidine bond etc. may be used, and various binding methods can be employed with reference to the literature (e.g. “Tanpakushitu IV Kouzoukino-sokan (Protein IV Correlation between structure and its function)” by the Japanese Biochemical Society; 1st edition Mar. 20, 1991, published by Tokyo Kagaku Dozin).

Reference to the above-mentioned preparation methods and known synthetic means in the related technical fields also enables the preparation of a conjugate of the present invention even where a polymer other than polyalkyleneimine is used.

Next, a method for introducing a protein into cells using a conjugate of the present invention will be described, but the method of the present invention is not limited thereto.

To a medium containing cells to which the protein is to be introduced, the conjugates or a solution containing the conjugates of the present invention is added. Thereafter cells are cultivated under appropriate conditions including culture temperature, culture period etc., and thereby the conjugates of the present invention are incorporated into cells. With the passage of time, the amount of the conjugates incorporated into cells is increased. According to the method of the present invention, the amount of protein to be introduced into cells can easily be controlled by changing the absolute amount, concentration, period for adding of the conjugates, and the like. Incidentally, it is assumed that the conjugates of the present invention can be incorporated into cells by a mechanism attributable to electrostatic interaction between the positively charged conjugates and a negatively charged cell surface. Thus, for incorporating the conjugates into cells in the medium, it is preferable to conduct the above process in the absence of anionic polymers such as heparin and nucleic acid. Further, the solution containing the conjugates of the present invention is inoculated directly on a living body by a method e.g. oral administration, intravenous administration, injection to an affected area, and dermal application, thus allowing conjugates to be incorporated into cells of the living body.

This specification includes part or all of the contents as disclosed in the specification and/or drawings of Japanese Patent Application No. 2001-363967, which is a priority document of the present application.

### BRIEF DESCRIPTION OF THE DRAWINGS

FIG. 1 is electropherogram of RNaseA-PEI conjugates of the present invention and native RNaseA.

FIG. 2 is a graph showing incorporation of RNaseA-PEI conjugates of the present invention into 3T3/SV40 cells and cell proliferation inhibition activity.

FIG. 3 is electropherogram of eGFP-PEI conjugates of the present invention, native eGFP and eGFP-ethylenediamine conjugate.

FIG. 4 is a graph showing comparison of fluorescence intensity among eGFP-PEI conjugates of the present invention, native eGFP and eGFP-ethylenediamine conjugate.

FIGS. 5(a)-(f) are observation views by fluorescence which show incorporation of eGFP-PEI conjugates of the present invention, native eGFP and eGFP-ethylenediamine conjugate into Balb/c3T3 A31K cells.

FIG. 6 is an observation view by fluorescence which shows expression of a reporter gene (eGFP) in T7-eGFP/COS-7 cells.

FIGS. 7(a)-(c) are observation views showing comparison of introduction efficiency of native eGFP, eGFP-PEI600 conjugate, and TAT-eGFP fusion protein into Balb/c3T3 cells.

### BEST MODE FOR EMBODYING THE INVENTION

The present invention will hereinafter be explained more in detail by way of Examples and Reference examples, but the scope of the present invention is limited in no way by the following Examples.

In the Examples, a number average molecular weight is determined by ebullioscopic method, and the amine values are determined by acid neutralization titration. A cation value is calculated using a number average molecular weight and the amine value determined as mentioned above.

In the following Examples, PEIs (Nippon Shokubai: tradename “EPOMIN”) having a number average molecular weight of 250, 600, 1000, 1200, and 1800 were used and they are described as PEI1250, PEI600, PEI1000, PEI1200, and PEI1800, respectively.

### (EXAMPLE 1)

Synthesis of RNaseA-PEI Conjugate

RNaseA, which is an enzyme that induces cell proliferation inhibition associated with protein synthesis inhibition with introduction into cells, was used as a protein.

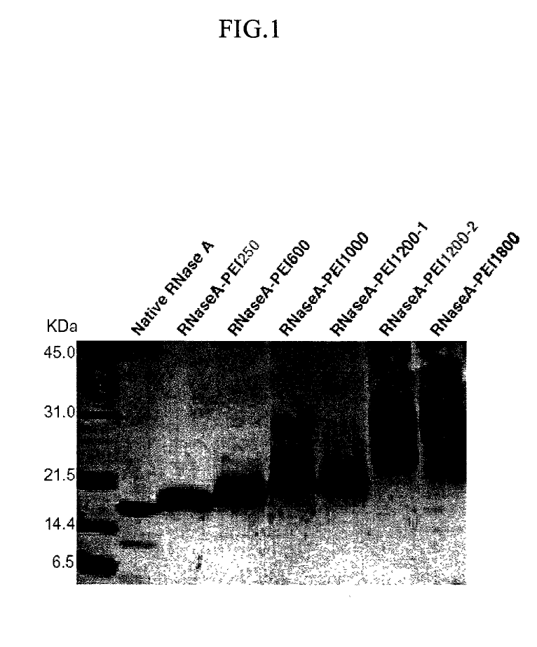

15 mg of RNaseA was dissolved in 1.5 ml of 60 mg/ml PEI aqueous solution (pH was adjusted to 5 by hydrochloric acid). 2.5 mg of EDC was added thereto, and the obtained solution was stirred for 16 hours at room temperature. After the reaction was completed, the reaction solution was dialyzed against water and finally purified by cationic ion exchange chromatography (CM-TOYOPEARL 650M) to give RNaseA-PEI conjugate (1 to 2 molecules of PEIs were bound per 1 molecule of RNaseA). About 1.5 μg of the obtained RNaseA-PEI conjugate was analyzed with 15% SDS-PAGE. The results are shown in FIG. 1.

Evaluation of Protein Function in RNaseA-PEI Conjugate

The hydrolytic rate of RNA catalyzed by RNase was measured using yeast RNA as a substrate, and the RNaseA-PEI conjugate was compared with native RNaseA in terms of enzyme activity. The relative activity of RNaseA-PEI conjugate to native RNaseA is shown in Table 1. In addition, the amine value and cation value of each PEI is also shown in Table 1. Here, RNaseA-PEI1200-1 and RNaseA-PEI1200-2 represent an RNaseA-PEI conjugate wherein about 1 and 2 molecules of PEI1200s were bound per 1 molecule of RNaseA, respectively. According to the results of Table 1, the conjugate of the present invention exhibited remarkably high enzyme activity in comparison with RNaseA-ethylenediamine prepared by a conventional method, and it was clear that the reduction of inherent function possessed by RNaseA was small.

TABLE 1

| Conjugate | Relative activity (%) |
|---|---|
| native RNaseA | 100 |
| RNaseA-PEI250 | 20.4 |
| RNaseA-PEI600 | 21.9 |
| RNaseA-PEI1000 | 28.0 |
| RNaseA-PEI1200-1 | 27.2 |
| RNaseA-PEI1200-2 | 8.9 |
| RNaseA-PEI1800 | 27.2 |
| RNaseA-ethylenediamine¹⁾ | 1.6 |

¹⁾about 8 ethylenediamines combined with 1 RNaseA

| | number | | |
|---|---|---|---|
| | average | | |
| | molecular | Amine value | Cation value |
| | weight | (mmol/g) | (Amine value × Mn)/1000 |
| | (Mn) | Found (Calcd.) | Found (Calcd.) |
| Etyhlene- | 60 | — (33) | — (2.0) |
| diamine | | | |
| PEI-100 | 100 | — (23) | — (2.3) |
| PEI-200 | 200 | — (23) | — (4.7) |
| PEI-250 | 250 | 21 (23) | 5.3 (5.8) |
| PEI-300 | 300 | 21 (23) | 6.3 (7.0) |
| PEI-600 | 600 | 20 (23) | 12 (14) |
| PEI-1000 | 1000 | 19 (23) | 19 (23) |
| PEI-1200 | 1200 | 19 (23) | 23 (28) |
| PEI-1800 | 1800 | 18 (23) | 32 (42) |
| PEI-3000 | 3000 | — (23) | — (70) |
| PEI-10000 | 10000 | 18 (23) | 180 (233) |
| PEI-20000 | 20000 | 18 (23) | 360 (460) |
| PEI-100000 | 100000 | — (23) | — (2330) |
| PEI-1000000 | 1000000 | — (23) | — (23300) |

Introduction of RNaseA-PEI Conjugate into Cells

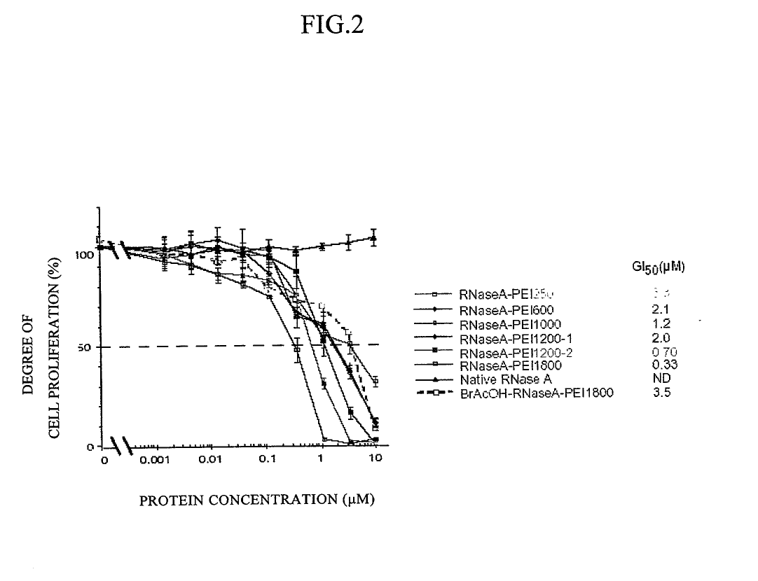

With respect to the above conjugates, cell proliferation inhibition activity against 3T3/SV40 cells was evaluated. In the cell proliferation inhibition activity experiment, a 96 well-plate was used. One thousand five hundred 3T3/SV40 cells per well were cultivated on DMEM+10% FBS medium for 12 hours. To the culture supernatant thereof, native RNaseA and RNaseA-PEI conjugates were added at various concentrations for each, and 3 days. Then, the cell proliferation degree was evaluated by MTT method. The results thereof are shown in FIG. 2. native RNaseA was not incorporated into cells and did not exhibit a significant cell proliferation inhibition activity. In contrast, the conjugates of the present invention were effectively introduced into cells and it was confirmed that the conjugates exhibited cell proliferation inhibition activity in proportion to the positive charge amount of the introduced PEIs.

### (EXAMPLE 2)

Synthesis of eGFP-PEI Conjugate

Enhanced Green Fluorescent Protein (eGFP available from CLONTECH) was used as a protein, thus allowing protein introduction into cells to be easily observed by fluorescence. The eGFP containing His tag at the amino terminal was expressed in Escherichia coli as a recombinant protein and purified.

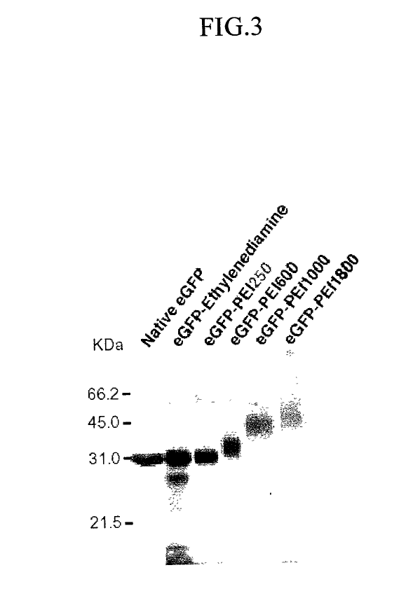

3 mg of eGFP was dissolved in 4.5 ml of 60 mg/ml PEI aqueous solution (adjusted to pH 5 with HCl). 10 mg of EDC was added thereto and the resultant solution was stirred for 30 seconds by a vortex mixer. Thereafter it was incubated at room temperature for 16 hours. The reaction solution was dialyzed against water, and finally purified by HiTrap(Ni⁺) chelate column to give an eGFP-PEI conjugate. The obtained eGFP-PEI conjugates were analyzed with 15% SDS-PAGE. The results are shown in FIG. 3.

Evaluation on Function of eGFP-PEI Conjugate

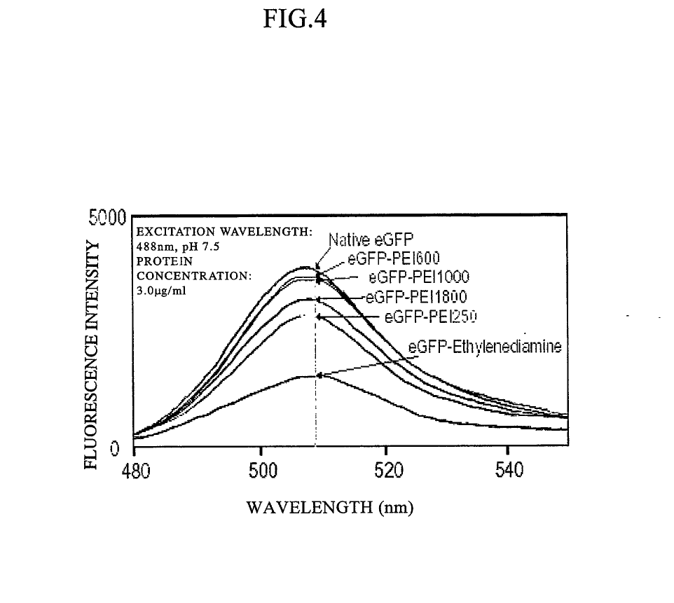

Native eGFP and the e-GFP-PEI conjugates of the present invention were compared with eGFP-ethylenediamine conjugate in terms of fluorescence intensity. Results are shown in FIG. 4. As can be shown in FIG. 4, in comparison with the conjugate multi-modified with ethylenediamine by a conventional method, the eGFP modified with about 1 PEI retained as high a fluorescence intensity as the native eGFP, and it was indicated that PEI modification does not exert a large influence on eGFP function.

Introduction of eGFP-PEI Conjugates into Cells

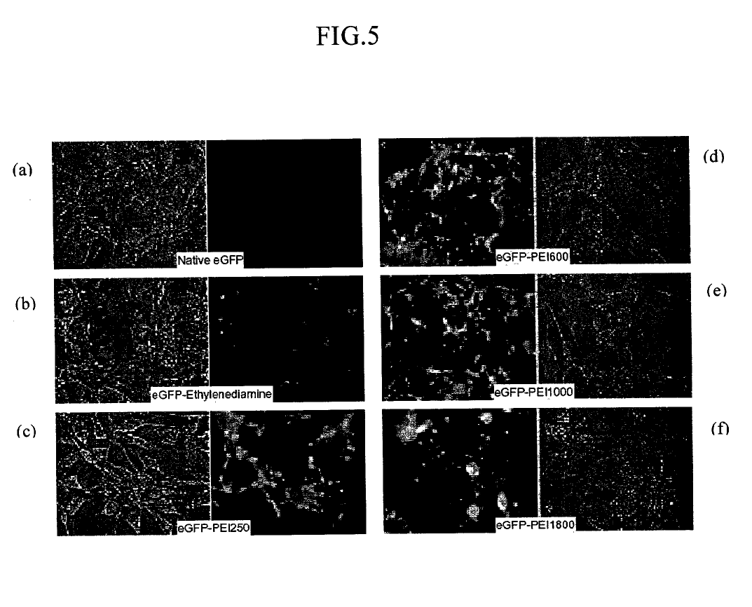

Balb/c3T3 A31K cells were cultivated on DMEM+10% FBS, 100 nM of eGFP-PEI conjugates (3 μg/ml) were added to the culture supernatant thereof. After 8 hours, incorporation of eGFP was observed by fluorescence. For comparison, tests on native eGFP and eGFP-ethylenediamine conjugate were carried out in the same manner as above. Results are shown in FIGS. 5(a)-(f). As can be shown in FIG. 5, there was almost no incorporation into cells for either native eGFP (FIG. 5(a)) or eGFP-ethylenediamine (FIG. 5(b)) conjugate, whereas the eGFP-PEI conjugates (FIGS. 5(c)-(f)) were effectively incorporated into cells. Incidentally, the fluorescence observation was conducted on live cells, and fixation etc. was not carried out.

### (EXAMPLE 3)

Synthesis of T7 RNA Polymerase-PEI1200 Conjugate

T7 RNA Pol. has a molecular weight of 100 kDa and 2 cystein residues are exposed on a molecular surface. A recombinant T7 RNA Pol. was expressed in E. coli. The obtained T7 RNA Pol. was purified, and a 4.62 mg/ml (46.8 μM) aqueous solution thereof was prepared. 0.107 ml of 100 mg/ml PEI1200 aqueous solution (adjusted to pH 8 with hydrochloric acid) and 1.4 mg/0.1 ml of SPDP ethanol solution were further mixed together (molar ratio, PEI1200:SPDP=2:1). This mixture solution was mixed with T7 RNA Pol. aqueous solution so that the molar ratio of SPDP to T7 RNA Pol. became 10:1, and then the obtained mixture was stirred for 16 hours at room temperature. After the reaction, gel filtration was conducted for purification, and consequently T7 RNA Pol.-PEI1200 conjugate wherein 2 molecules of PEI1200 were bound per 1 molecule of T7 RNA Pol. was obtained.

Expression of a Reporter Gene (eGFP) by Introducing T7 RNA Pol.-PEI1200 into Cells

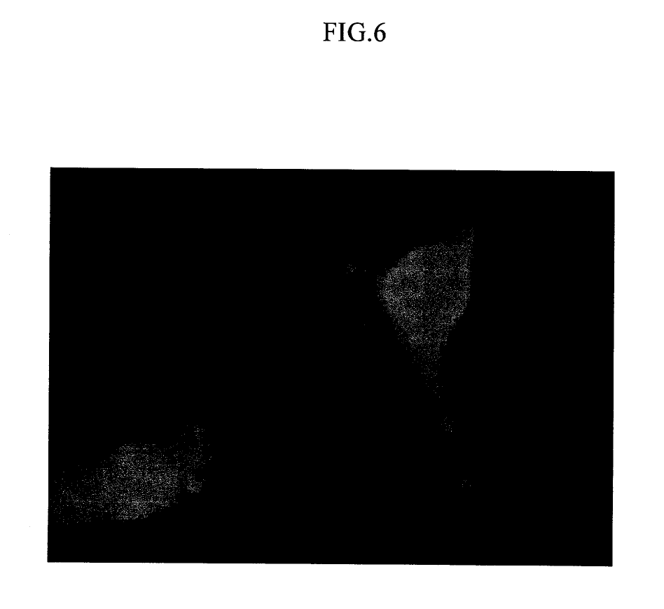

A plasmid DNA having an eGFP gene connected downstream of a T7 promoter was introduced into a COS-7 cell by electroporation for preparing a T7-eGFP/COS-7 cell. 1 μM of T7 RNA Pol.-PEI1200 was added to the culture solution of T7-eGFP/COS-7 cells (DMEM+10% FBS medium), and the mixture was cultivated for 1 day at room temperature. After cultivation, eGFP expression was observed by fluorescence. FIG. 6 shows an observation view thereof. In contrast, without the addition of T7 RNA Pol.-PEI1200, it was confirmed that the eGFP gene was not expressed.

### (EXAMPLE 4)

Comparison of Cell Introduction Efficiency by eGFP-PEI600 Conjugate and TAT-eGFP Fusion Protein

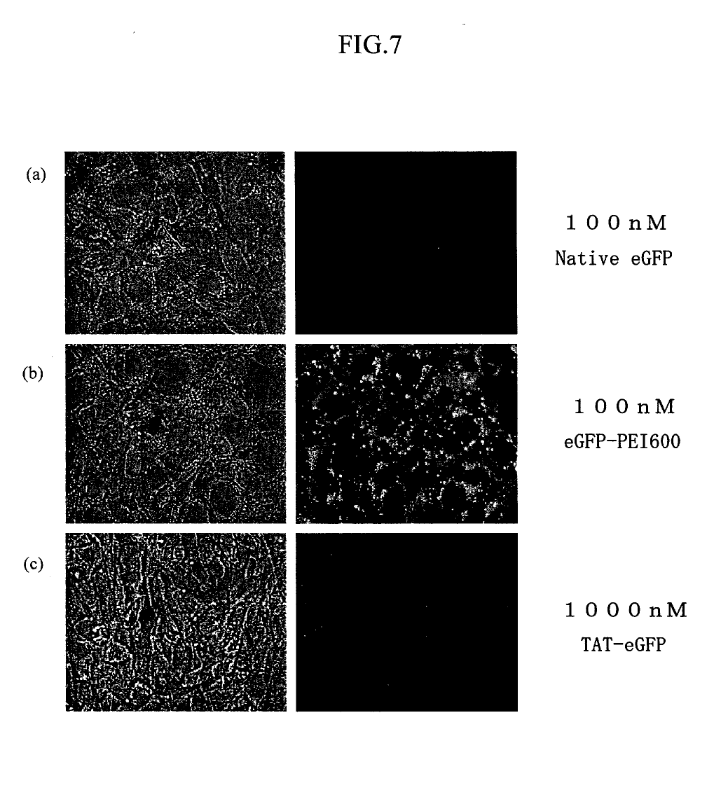

100 nM of the eGFP-PEI600 conjugate synthesized in accordance with Example 2 was added to 10% FBS+DMEM medium containing Balb/c3T3 cells, and cultivated for 6 hours at 37° C. After cultivation, incorporation of eGFP into cells was observed by fluorescence. The TAT peptide is a peptide having an amino acid sequence of GRKKRRQRRRG, and it is reported that the protein can be incorporated easily into cells by conjugating it with a protein. The results are shown in FIGS. 7(a)-(c). According to the results shown in FIG. 7, when the TAT-eGFP fusion protein was used, almost no protein was incorporated into cells (FIG. 7(c)). In contrast, when the eGFP-PEI600 conjugate of the present invention was used, even at a concentration of one-tenth of TAT-eGFP fusion protein, the protein was incorporated into cells with high efficiency (FIG. 7(b)).

### (EXAMPLE 5)

Synthesis of a RNaseA-PEI600 Conjugate Using 2-iminothiolane

10 mg (0.73 μmol) of RNaseA was dissolved in 500 μl of 0.1 M phosphate buffer solution (pH 8). 0.5 mg (3.6 μmol) of 2-iminothiolane was added thereto and the mixture solution was stirred for 15 minutes at room temperature. Then, 10 mg (36 μmol) of BrCH₂CONHNHCOCH₂Br was added, and dispersed with ultrasonic. Then the obtained solution was stirred for 1 hour at room temperature. After the reaction was completed, the reaction solution was centrifuged. The supernatant thereof was mixed with 0.5 ml (83 μmol) of 10% PEI solution (adjusted to pH 8), and stirred for 1 hour at room temperature. After the reaction was completed, the reaction solution was dialyzed against water to give a RNaseA-PEI600 conjugate.

The thus obtained conjugate was tested in the same manner as in Example 1 with respect to cell proliferation inhibition against 3T3/SV40 cells. It was confirmed that the RNaseA-PEI600 conjugate obtained by using 2- iminothiolane as well as the RNaseA-PEI600 conjugate obtained by using EDC were both effectively incorporated into cells and exhibited cell proliferation inhibition activity.

Industrial Applicability

According to the present invention, there is provided a method for introducing a protein into cells without serious loss of the protein function and with remarkably high efficiency and time and amount controllability.

Namely, the gene introduction method has conventionally been the mainstream method for enabling a designated protein to function in cells, and this method is suitable for constant expression and functioning in cells of the protein. In contrast, a method for introducing a protein into cells according to the present invention is suitable for transient functioning of the protein. This type of a novel technique can be a means for function analysis of a protein whose function is unknown. Furthermore, this technique can be a means for solving various problems with which the conventional gene introduction method is burdened.

Moreover, in comparison with the conventional method of cationization by ethylenediamine which requires modification of many side chains within a protein molecule, since the method of the present invention enables many cations to be introduced by modifying a small number of side chains i.e. within the range of one to several side chains, it exerts slight influence on the structure and function of the protein.

All the publications and patent applications cited herein are incorporated by reference in their entirety.

What is claimed is:

1. A method for transducing a protein or peptide into a cell, comprising a step of transporting the protein or peptide into the cell by using a conjugate formed by binding the protein or peptide with a polymer having a cation value of more than 2 and no more than 30,000.

2. The method according to claim 1, wherein the polymer is a polymer having a polyalkylenepolyamine skeleton, a polyallylamine skeleton, a polyvinylamine skeleton, a poly(dialkylaminoalkyl (meth)acrylate) skeleton, a poly(meth) acrylic dialkylaminoalkylamide skeleton, a polyamidine skeleton, a polyvinylpyridine skeleton or polyvinylimidazole skeleton, or a salt thereof.

3. The method according to claim 1, wherein the polymer has a number average molecular weight within a range of from 100 to 1,000,000.

4. The method according to claim 1, wherein the conjugate is formed by binding the protein or peptide with the polymer via covalent bond.

5. The method according to claim 1, wherein the conjugate is formed by binding the protein or peptide with the polymer via an amide bond, disulfide bond or thioether bond.

6. A method for transducing a protein or peptide into a cell, comprising a step of transporting the protein or peptide into the cell by using a conjugate formed by binding the protein or peptide with a polymer having a polyalkylenepolyamine skeleton.

7. The method according to claim 6, wherein the polymer is polyalkyleneimine.

8. The method according to claim 6, wherein the polymer is polyethyleneimine.

9. The method according to claim 6, wherein the polymer has a number average molecular weight within a range of from 100 to 1,000,000.

10. The method according to claim 6, wherein the polymer has a number average molecular weight within a range of from 100 to 100,000.

11. The method according to claim 6, wherein the conjugate is formed by binding the protein or peptide with the polymer via covalent bond.

12. The method according to claim 6, wherein the conjugate is formed by binding the protein or peptide with the polymer via an amide bond, disulfide bond or thioether bond.

13. A conjugate formed by binding a protein or a peptide with a polymer having a cation value of more than 2 and no more than 30,000, and having a number average molecular weight within a range of from 100 to 1,000,000.

14. The conjugate according to claim 13, wherein the polymer is a polymer having a polyalkylenepolyamine skeleton, a polyallylamine skeleton, a polyvinylamine skeleton, a poly(dialkylaminoalkyl (meth)acrylate) skeleton, a poly(meth) acrylic dialkylaminoalkylamide skeleton, a polyamidine skeleton, a polyvinylpyridine skeleton or polyvinylimidazole skeleton, or a salt thereof.
